(19) United States
(12) Patent Application Publication
(10) Pub. No.: US 2004/0051053 A1
(43) Pub. Date: Mar. 18, 2004

(54) Universal pattern generator with multiplex addressing

(76) Inventors: William A. Barletta, Oakland, CA (US); Ka-Ngo Leung, Hercules, CA (US)

Correspondence Address: LAWRENCE BERKELEY NATIONAL LABORATORY, ONE CYCLOTRON ROAD, MAIL STOP 90B, UNIVERSITY OF CALIFORNIA, BERKELEY, CA, 94720, US

(21) Appl. No.: 10/443,574

(22) Filed: May 22, 2003

Related U.S. Application Data
(60) Provisional application No. 60/382,672, filed on May 22, 2002.

(51) Int. Cl. A61N 5/00; G21G 5/00

(52) U.S. Cl. 250/492.1

(57) ABSTRACT

A maskless micro-ion-beam reduction lithography (MMRL) system generates patterns of beamlets by switching individual beamlets on or off using a universal pattern generator which is positioned as the extraction electrode of the plasma source. Each aperture of the pattern generator is independently controlled to pass a beamlet. A multiplex addressing system to the individual apertures of the MMRL system is used to reduce the number of electrical connections. An additional layer of control electrodes is added. All apertures in each row of a first layer are connected to a single row address line. All apertures in each column of a second layer are connected to a single column address line. By using the combination of row and column lines, each aperture can be controlled.

### RELATED APPLICATIONS

This application claims priority of Provisional Application Ser. No. 60/382,672 filed May 22, 2002, which is herein incorporated by reference.

### BACKGROUND OF THE INVENTION

The invention relates generally to ion beam lithography and more particularly to ion beam lithography systems without stencil masks.

As the dimensions of semiconductor devices are scaled down in order to achieve ever higher levels of integration, optical lithography will no longer be sufficient for the needs of the semiconductor industry, e.g. DRAM and microprocessor manufacture. Alternative “nanolithography” techniques will be required to realize minimum feature sizes of 0.1 μm or less. In addition, the next generation lithography technologies must deliver high production throughput with low cost per wafer. Therefore, efforts have been intensified worldwide in recent years to adapt established techniques such as X-ray lithography, extreme ultraviolet lithography (EUVL), electron-beam (e-beam) lithography, and ion projection lithography (IPL), to the manufacture of 0.1 μm-generation complementary metal-oxide-semiconductor (CMOS) technology. Significant challenges exist today for each of these techniques. In particular, there are issues with complicated mask technology.

Conventional ion projection lithography (IPL) systems require many stencil masks for semiconductor circuit processing. An ion source with low energy spread is needed to reduce chromatic aberration. A small beam extracted from the source is accelerated and expanded to form a parallel beam before impinging onto a large area stencil mask which contains many small apertures. The aperture pattern is then projected onto a resist layer on a wafer after the beam is reduced in size and made parallel by an Einzel lens system. Different masks with particular patterns must be used for each layer to be formed on the wafer.

In the conventional IPL setup, the stencil mask is extremely thin, e.g. about 3 μm, to minimize beam scattering inside the aperture channels. Since the beam energy is high, about 10 keV, when it arrives at the mask, both sputtering and mask heating will occur, causing unwanted mask distortion and instability.

An alternative IPL system, the plasma-formed IPL system, eliminates the acceleration stage between the ion source and stencil mask. Instead a much thicker and more stable mask is used as a beam forming electrode, positioned next to the plasma in the ion source. The extracted beam passes through an acceleration and reduction stage onto the resist coated wafer. Because low energy ions, about 30 eV, pass through the mask, heating, scattering, and sputtering are minimized. However, a separate mask is needed for each new feature pattern to be projected onto the wafer.

### SUMMARY OF THE INVENTION

Accordingly it is an object of the invention to provide an ion projection lithography (IPL) system which has no stencil mask.

It is also an object of the invention to provide an IPL system which can generate a variety of different beam patterns using a single apparatus.

It is another object of the invention to provide an efficient addressing system for the beamlet generator of such an IPL system.

The invention is an addressing system for a maskless micro-ion-beam reduction lithography (MMRL) system which produces feature sizes down to 0.1 μm or less. The MMRL system operates without a stencil mask. The patterns are generated by switching individual beamlets on or off using a universal pattern generator which is positioned as the extraction electrode of the plasma source. Each aperture of the pattern generator is independently controlled to pass a beamlet. The pattern generator is a two electrode blanking system. A multicusp ion source with magnetic filter produces ion beams with low energy spread, as low as 0.6 eV. The low energy plasma ions are selectively passed through the pattern generator by applying suitable voltages to the electrodes to produce the desired pattern. A beam accelerator and reduction column after the pattern generator produces a demagnified pattern on the resist. The MMRL system is described in U.S. patent application Ser. No. 09/289,332, which is herein incorporated by reference.

The invention provides a multiplex addressing system to the individual apertures of the MMRL system to reduce the number of electrical connections. An additional layer of control electrodes is added. All apertures in each row of a first layer are connected to a single row address line. All apertures in each column of a second layer are connected to a single column address line. By using the combination of row and column lines, each aperture can be controlled.

### BRIEF DESCRIPTION OF THE DRAWINGS

FIG. 1 shows an MMRL system.

FIGS. 2, 3 are sectional and perspective views of a two electrode pattern generator for the MMRL system.

FIG. 4 is a cross-sectional view of the electrodes associated with an aperture of a universal pattern generator having a multiplexed addressing system of the invention.

FIG. 5 shows the row and column connections to an array of beamlet forming apertures in a multiplexed addressing system of the invention.

FIGS. 6A, B provide a general and a layer view of the X-Y multiplexing system of the invention.

FIG. 7 is a graph of transmission currents due to varying switching voltages.

FIGS. 8A, B show the electron beam current for on and off conditions.

### DETAILED DESCRIPTION OF THE INVENTION

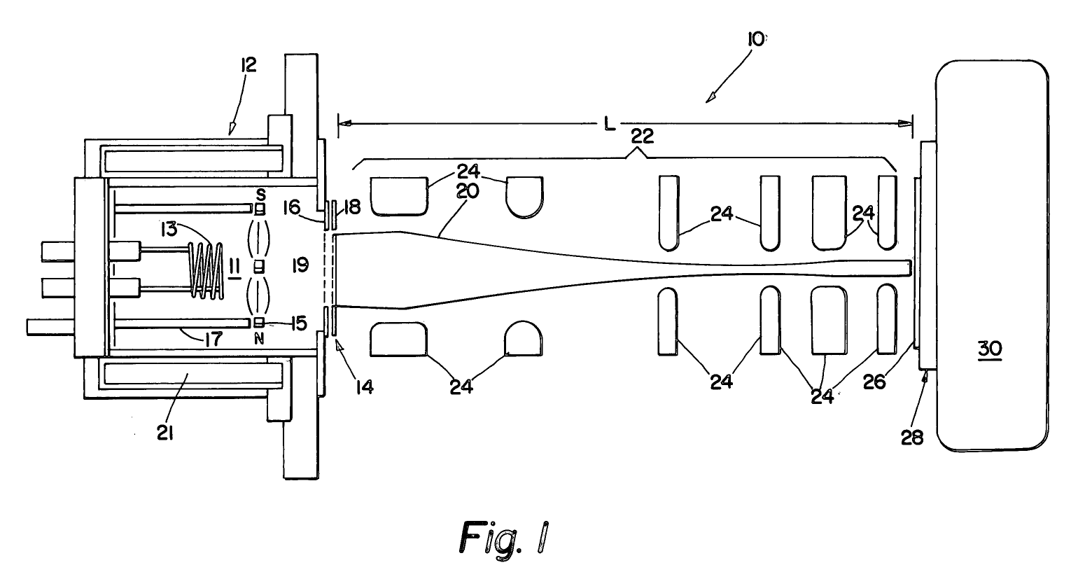

A maskless micro-ion-beam reduction lithography (MMRL) system 10, shown in FIG. 1, has an ion source 12 with a pattern generator 14 formed of a pair of electrodes 16, 18 positioned to form a multi-beamlet ion beam 20. The extracted beam 20 passes through an acceleration and reduction column 22, of length L, formed of a plurality of electrode lenses 24. Column 22 reduces the micro-beamlet pattern produced by pattern generator 14 by factors greater than 5 to achieve feature sizes less than 100 nm. The beam from column 22 is incident on a resist layer 26 on a wafer 28 which is mounted on a mechanical stage or support 30. The wafer 28 with exposed resist layer 26 is processed by conventional techniques.

The MMRL system 10 is made up of the following major components:

A. Ion Source—Multicusp Plasma Generator

As shown in FIG. 1, ions are produced in a plasma generation region 11 of an ion source 12 which may be of conventional design. Plasma is generated by an RF antenna 13 or alternatively by a filament. A linear magnetic filter 15 or a coaxial magnetic filter 17 is used to decrease energy spread of the ions. The plasma ions pass to extraction region 19 of source 12. Conventional multicusp ion sources are illustrated by U.S. Pat. Nos. 4,793,961; 4,447,732; 5,198,677, which are herein incorporated by reference. U.S. Pat. No. 6,094,012, which is herein incorporated by reference, describes a preferred ion source with a coaxial magnetic filter which has a very low energy spread.

The multicusp plasma generator provides positive ions needed for resist exposure. Normally either hydrogen or helium ions are used for this purpose. The external surface of ion source 12 is surrounded by columns of permanent magnets 21 which form multicusp fields for primary ionizing electron and plasma confinement. The cusp fields are localized near the source wall, leaving a large portion of the source free of magnetic fields. As a result, this type of ion source can generate large volumes of uniform and quiescent plasmas having relatively flat radial density profiles. For example, a 30 cm diameter chamber can be used to form a uniform plasma volume of about 18 cm diameter. Larger uniform plasmas can be generated by using bigger source chambers with well designed permanent magnet configurations.

The plasma of the multicusp source can be produced by either radio-frequency (RF) induction discharge or by dc filament discharge. However for MMRL, an RF driven discharge is preferred since the quartz antenna coil typically used for antenna 13 will not generate impurities and there is no radiation heating of the first electrode of column 22 due to hot tungsten filament cathodes. The discharge plasma will be formed in short pulses, e.g. about 300 ms pulse length, with high or low repetition rates. With a magnetic filter in the source, the axial ion energy spread can be reduced to values below 1 eV. The output current density is high, e.g. greater than 250 mA/cm2, for pulsed operation and the source can produce ion beams of nearly any element.

B. Pattern Generator—Multibeamlet Extraction System

The open end of ion source 12 is enclosed by pattern generator 14 which forms a multibeamlet extraction system. Pattern generator 14 is formed of a spaced pair of electrodes 16, 18 and electrostatically controls the passage of each individual beamlet to form a predetermined beamlet pattern to be projected.

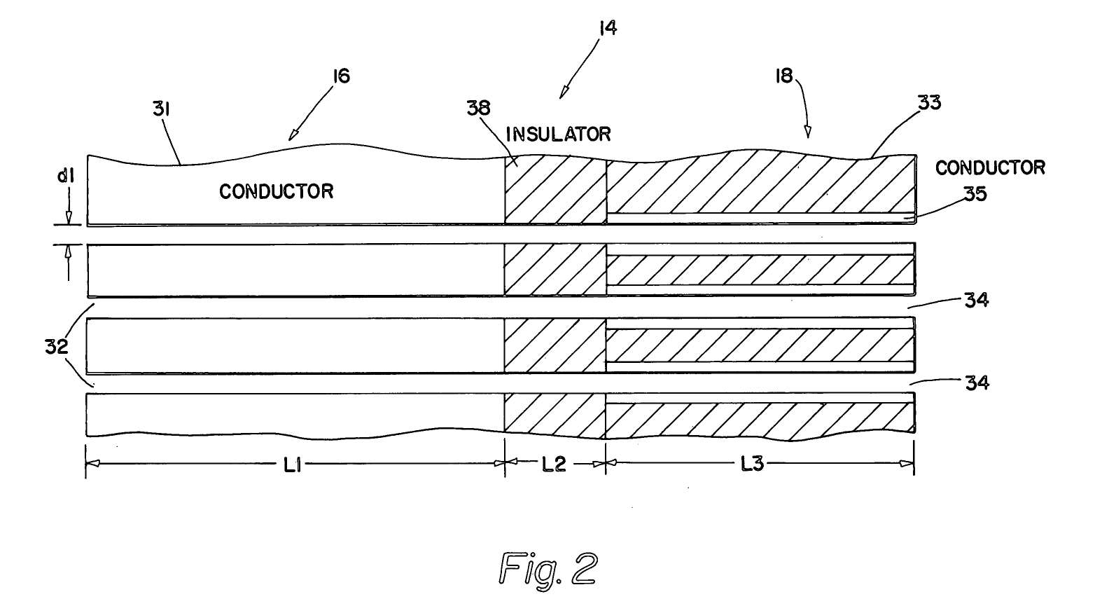

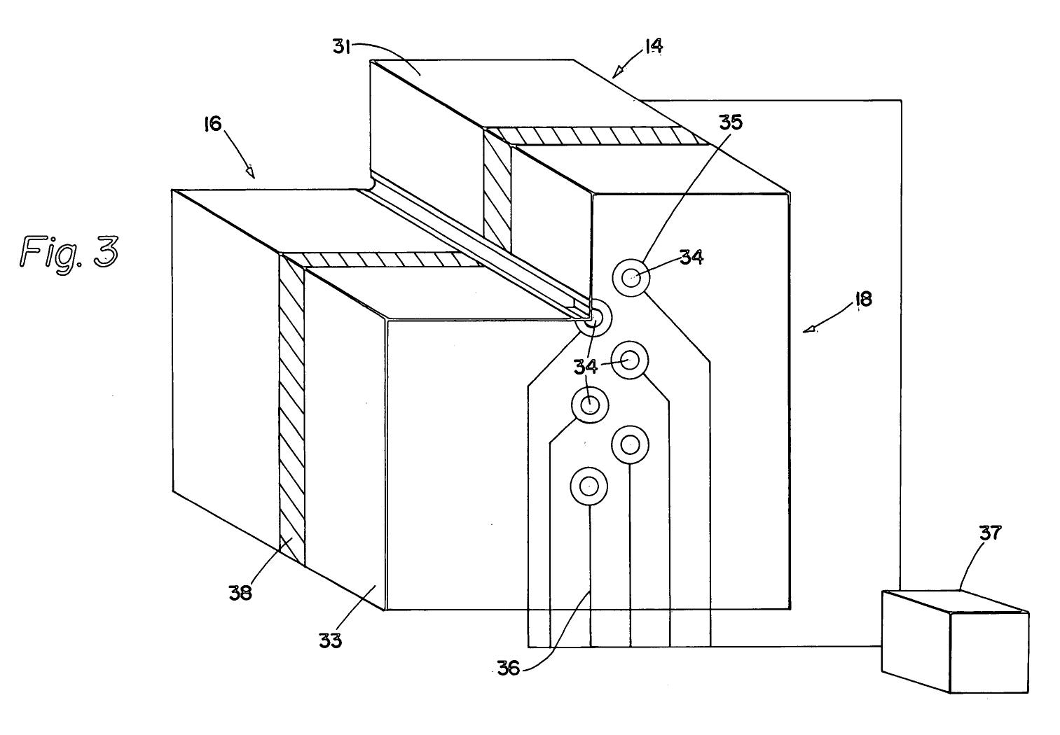

FIGS. 2, 3 illustrate a preferred embodiment of a pattern generator—beamlet extractor 14. First electrode 16 is the plasma or beam forming electrode and is formed of a conductor 31 having a plurality of apertures or channels 32 formed therein. The apertures 32 on the extractor 14 will be arranged to fall within the uniform plasma density region of the source. Second electrode 18 is the extraction or beamlet switching electrode and is formed of an insulator 33 having a plurality of apertures or channels 34 formed therein. Each channel 34 contains an annular conductor 35 which is electrically connected by electrical connection 36 to a programmable voltage source 37 which can apply different voltages to each of the annular conductors 35. Conductor 31 is also connected to voltage source 37 or to a separate source. Electrodes 16, 18 are separated by an insulator 38. Channels 32, 34 are aligned with each other and extend through insulator 38. Conductor 31, insulator 38, and insulator 33 have thickness of L1, L2, L3 respectively. Typical values are L1=20 μm, L2=5 μm, and L3=15 μm, for a total thickness of about 40 μm which is much thicker than the thickness of a typical stencil mask. The diameter of the channels 32, 34 through the pattern generator 14 is d1, typically about 1 μm.

In operation, the first electrode is biased negatively, about 30 V, with respect to the ion source chamber wall. A very thin plasma sheath is formed parallel to the first electrode surface. Positive ions in the plasma will fall through the sheath and impinge perpendicular to the electrode with an energy of about 30 eV. Ions will enter the apertures of the first electrode forming multiple beamlets. With such low impact energies, sputtering of the electrode will not occur. In addition, the heating power generated by ions on the electrode is extremely small and will not produce any instability of the extraction system. Because of low incoming energy, ion scattering inside the aperture channels is minimized. The ions will be absorbed on the channel surfaces rather than forming aberrated beams as they leave the apertures.

In the second electrode, if the annular conductors surrounding each aperture channel are also biased at the same potential as the first electrode, then ions will leave the apertures with an energy of about 30 eV. However, if the annular conductors of the second electrode are biased positively with respect to the first electrode, then the flow of ions to the aperture exit will be impeded by the electrostatic field. If this bias voltage is high enough, then the beam output will essentially become zero, i.e. the beam is turned off. Since the voltage on each annular conductor of the second electrode can be independently controlled, each individual beamlet can independently be turned on and off. Thus any desired beamlet pattern can be produced by the pattern generator, and the pattern can easily be switched to a different pattern.

In this multibeamlet extraction system, circular apertures will typically be employed. There will be many apertures, e.g. each with a diameter of about 1 μm and a separation less than 100 nm. These circular patterns will be projected onto the resist on the wafer with a reduction factor of typically 20. The final image size of each beamlet will then be 50 nm with separation less than 5 nm. The material between the image dots will be made so small that they will disappear during the etching process.

C. Acceleration and Beam Reduction Column

The micro-ion-beams leave the apertures of the extractor 14 with an energy of about 30 eV. They will be further accelerated and focussed by a simple all electrostatic acceleration and reduction column (lens system) 22 which is made up of a plurality of electrodes 24. The final parallel beam can be reduced to different sizes according to the particular lens design. The total length of one accelerator/reduction column is only about 65 cm, and other designs may be even shorter, e.g. about 35 cm. The beam reduction system can be designed with or without beam crossover.

A portion of the acceleration and reduction column 22 may be made up of an Einzel lens system which includes a pair of split electrodes. The two Einzel electrodes can be used to steer the beamlets by applying suitable voltages. This feature is important for circuit stitching purposes when the field of exposure is smaller than the chip size. By applying different voltages on the segments of the split electrodes, one can steer or scan the beam very fast, as fast as several cm in tens of nanoseconds, in the x or y direction.

D. Multiplex Addressing System

In the universal pattern generator, each aperture of the pattern generator is independently controlled to pass a beamlet. A wire to each control electrode is provided. However, as the number of apertures increases, e.g. an M×N array, MN wires are needed, creating a difficult fabrication problem.

Using a multiplex addressing approach, an M×N array only requires M+N wires (instead of MN wires). An additional layer of control electrodes is added, separated from the first layer by an insulator. All apertures in each row of the first layer are connected to a single row address line. All apertures in each column of the second layer are connected to a single column address line. By using the combination of row and column address lines, each aperture can be controlled. The electrodes of the second layer can be split electrodes for beamlet steering.

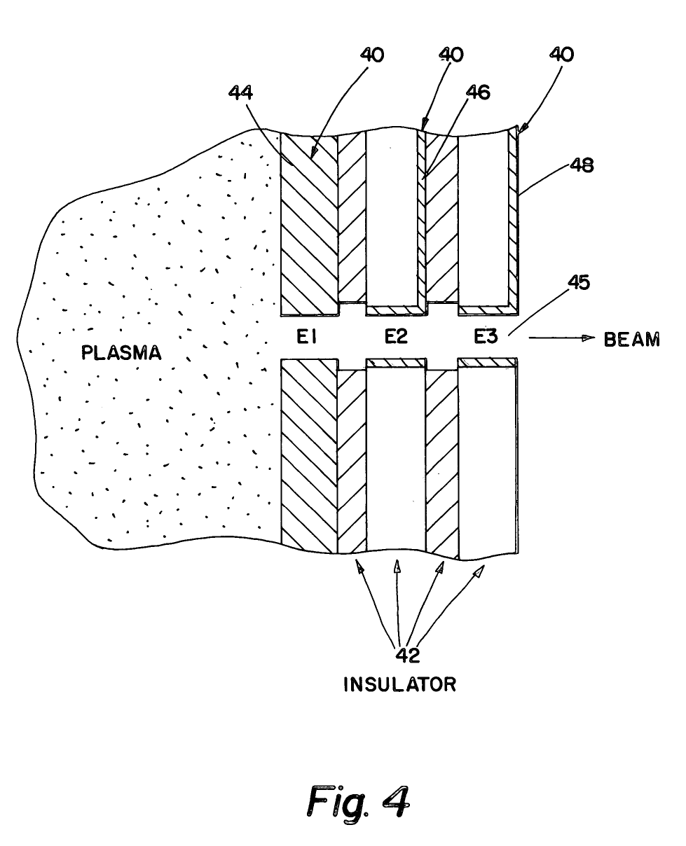

The electrode structure for multiplex addressing is shown in FIG. 4. The different conductive layers or electrodes (E1), (E2), (E3) formed of conductors 40 separated by insulators 42 are used. The first electrode 44 is the plasma or beam forming electrode, similar to electrode 31 in FIGS. 2, 3. The single switching electrode 35 of FIGS. 2, 3 is replaced by a pair of control electrodes 46, 48. The first control electrode 46 may be connected to the row address line and the second control electrode 48 may be connected to the column address line. Aperture 45 is formed through electrodes E1, E2, E3, through which an ion beam is extracted.

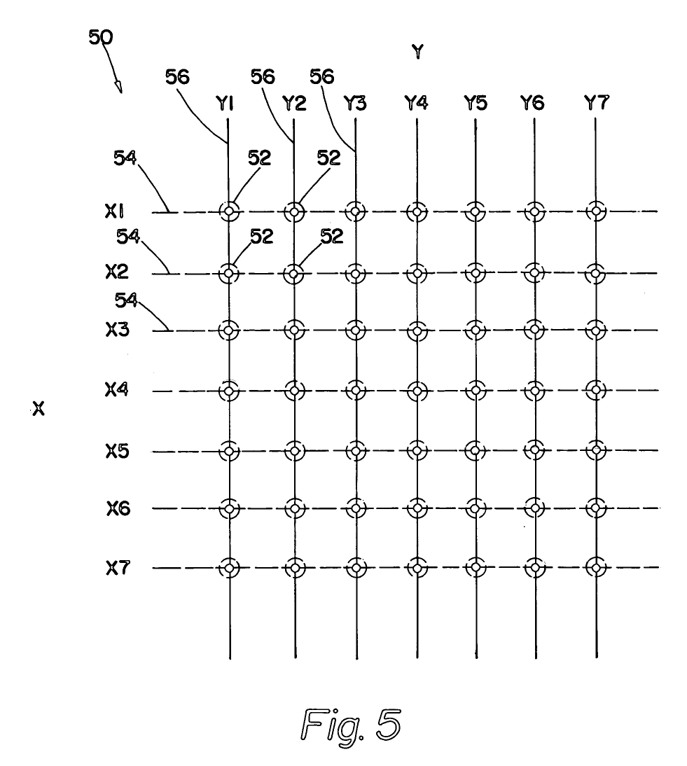

As shown in FIG. 5, an array 50 of universal mask extraction apertures 52, each with an electrode structure as shown in FIG. 4, is connected to a plurality (e.g. 7) of row address lines 54 (X1 . . . X7) and column address lines 56 (Y1 . . . Y7).

Different writing schemes can be used with the MMRL technique. The entire patternable surface can be filled with switchable apertures. But since each switching element requires an electrical connection, the number of connectors would be 10¹² for a 10⁶×10⁶ aperture arrangement. A more realistic scheme is to combine the switching with either beam or mechanical scanning of the wafer.

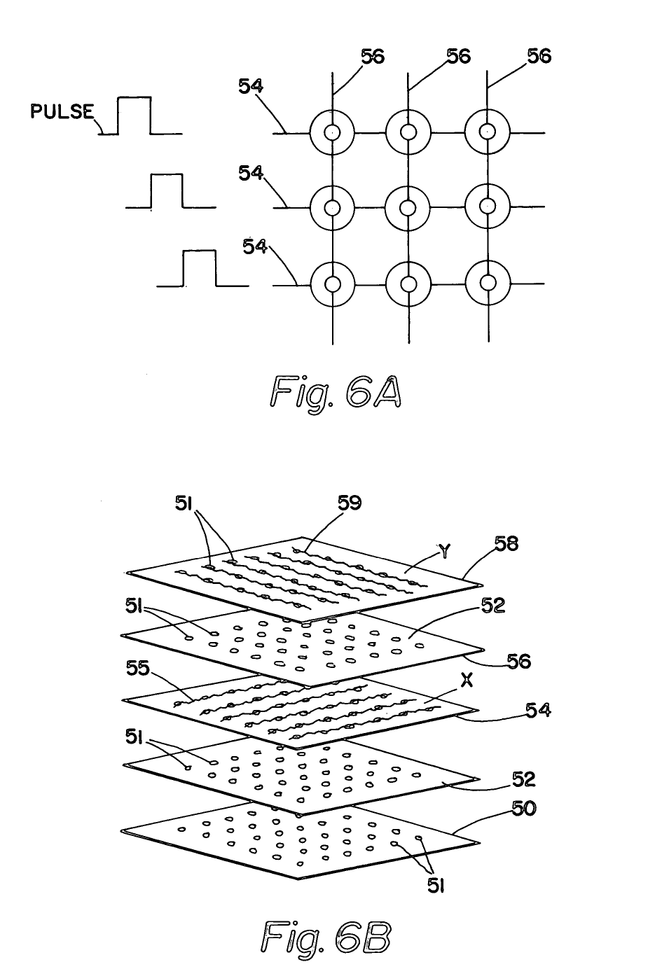

There is another way of reducing the number of connections to the pattern generator. By adding another layer to the pattern generator, it is possible to perform simple X-Y addressing via multiplexing as illustrated in FIGS. 4, 5 and in FIGS. 6A, B. FIG. 6A shows a sequence of pulses being applied to (three) row address lines 54; pulses can similarly be applied to column address lines 56. FIG. 6B shows an exploded view of the layer structure for a multiplexed addressing system. Conductor layer 50, containing a plurality of apertures 51, is the plasma or beam forming electrode (corresponding to electrode 44 in FIG. 4). The next layer is an insulator layer 52, which also includes a plurality of apertures 51. Next is the row (X) addressing layer 54, which is formed of an insulator and also includes a plurality of apertures 51. Each aperture 51 on layer 54 includes an electrode structure similar to electrode 46 in FIG. 4, with all electrodes in a row connected to a row address line 55. The next layer is an insulator layer 56, which also includes a plurality of apertures 51. Finally is the column (Y) addressing layer 58, which is formed of an insulator and also includes a plurality of apertures 51. Each aperture 51 on layer 58 includes an electrode structure similar to electrode 48 in FIG. 4, with all electrodes in a column connected to a column address line 59. The layers 50, 52, 54, 56, 58 are assembled together with all apertures 51 aligned so that ion beamlets can be extracted. Each beamlet is addressed by a combination of row and column.

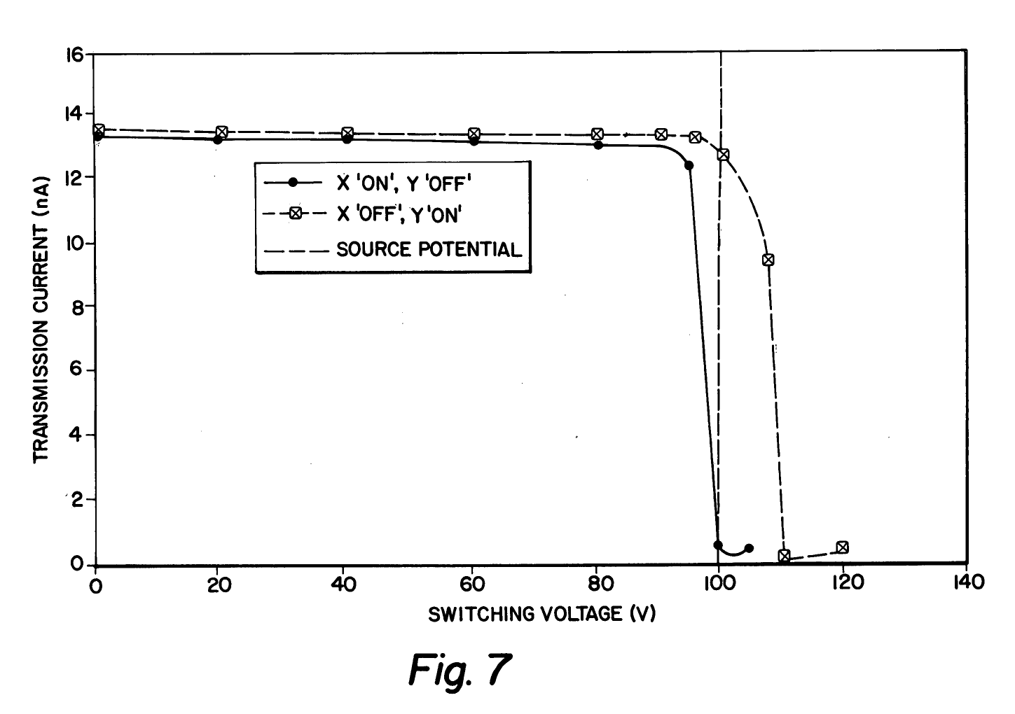

In this case, for an array of 10⁶×10⁶ apertures, only 2×10⁶ connections would be required. Either X or Y voltages can be used to turn the beam off. The bias voltage required to turn the beam off is 1-3 V more positive with respect to the source potential. The only time the beam is on is when X and Y are below the source potential as shown in FIG. 7. Although it seems that the first switching electrode is more effective in switching the beam off, the multiplexing method can also be used.

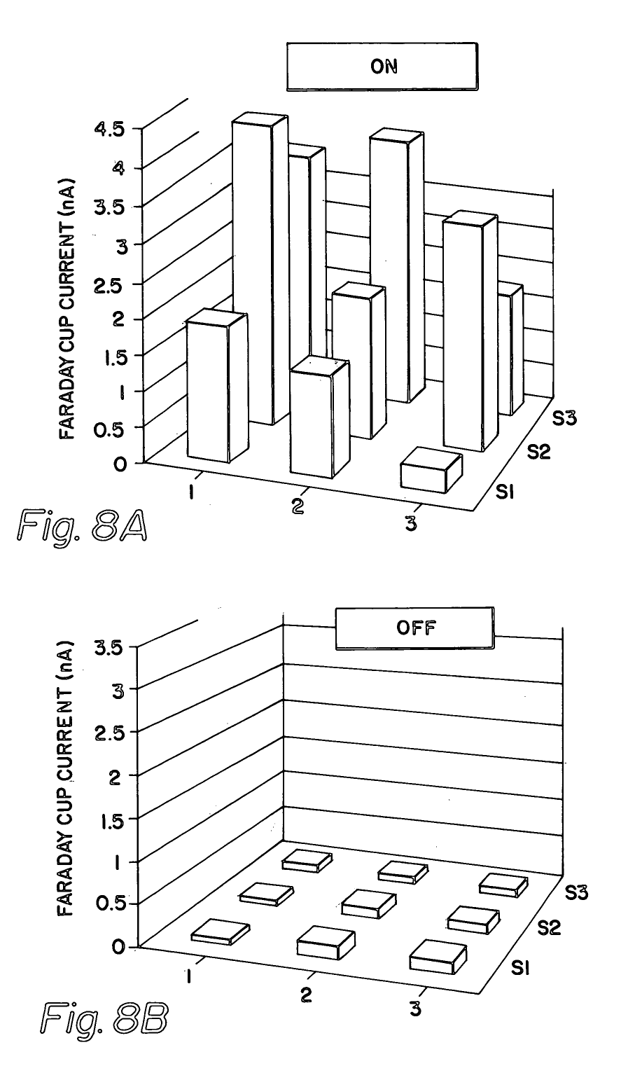

The same setup can be used for electron beam switching. The polarity of the power supplies was reversed to extract electrons. Source operation and discharge conditions remain the same with argon used as the working gas. However, other source gases can also be used selectively. FIG. 8 shows the electron beam current for the on and off conditions. Under the same conditions, the electron beam current is higher than the ion beam current.

The MMRL system uses a pattern generator which electrostatically produces and manipulates, i.e. switches on and off, a plurality of micro-ion beamlets which are coupled to a beam reduction and acceleration column. A compact addressing scheme uses multiplexed row and column lines to control each of the beamlets. Beam demagnification factors of up to 50 or more can be achieved with simple all-electrostatic accelerator columns. The system can provide economic and high throughput processing.

Thus the invention provides method and apparatus for ion beam projection lithography which could be used in semiconductor manufacturing with minimum feature sizes of 100 nm or less. Multicusp ion sources with magnetic filters produce uniform plasma volumes larger than 20 cm in diameter. By employing a patterned beamlet switching system, in which each beamlet is individually controlled, as the extractor for the ion source, a beam with a desired feature pattern is produced without requiring a separate mask for each pattern. The beam with selected pattern is then passed through a compact all electrostatic column to demagnify the feature pattern to a desired level.

Changes and modifications in the specifically described embodiments can be carried out without departing from the scope of the invention which is intended to be limited only by the scope of the appended claims.

1. A pattern generator for controllably producing a plurality of micro-ion-beamlets from an ion source, comprising:
a first electrode positioned adjacent to the ion source and having a first plurality of apertures formed therein for producing an array of micro-ion beamlets by passing ions from the ion source therethrough;
a second electrode layer in a spaced relation to the first electrode and having a second plurality of apertures formed therein and aligned with the first plurality of apertures, the second electrode layer having an electrode at each aperture for electrostatically and individually controlling the passage therethrough of each of the micro-ion-beamlets passing through the first electrode;
a third electrode layer in a spaced relation to the second electrode layer and having a third plurality of apertures formed therein and aligned with the first and second plurality of apertures, the third electrode layer having an electrode at each aperture for electrostatically and individually controlling the passage therethrough of each of the micro-ion-beamlets passing through the first electrode and second electrode layer;
a multiplex addressing system connected to the electrodes in the second and third electrode layers.

2. The pattern generator of claim 1 wherein the first electrode comprises a conductor having the first plurality of apertures formed therein.

3. The pattern generator of claim 2 wherein the second and third electrode layers each comprise an insulator having the second and third plurality of apertures formed therein, and a conductive electrode element at each aperture.

4. The pattern generator of claim 3 wherein the multiplex addressing system further comprises a plurality of row address lines, each row address line connected to each conductive electrode element in a row of apertures of the second electrode layer, and a plurality of column address lines, each column address line connected to each conductive electrode element in a column of apertures of the third electrode layer.

5. A maskless micro-ion-beam reduction lithography (MMRL) system, comprising:
a plasma generator which produces ions in a plasma generation region;
a pattern generator positioned adjacent to the plasma generation region of the ion source for electrostatically producing a controlled pattern of micro-ion-beamlets;
a multiplex addressing system connected to the pattern generator;
an acceleration and reduction column following the pattern generator and having aligned apertures therethrough for accelerating and focusing the micro-ion-beamlets extracted from the plasma generation region to produce a demagnified final ion beam.

6. The MMRL system of claim 5 wherein the plasma generator comprises a multicusp ion source.

7. The MMRL system of claim 5 wherein the pattern generator comprises:
a first electrode positioned adjacent to the ion source and having a first plurality of apertures formed therein for producing an array of micro-ion beamlets by passing ions from the ion source therethrough;
a second electrode layer in a spaced relation to the first electrode and having a second plurality of apertures formed therein and aligned with the first plurality of apertures, the second electrode layer having an electrode at each aperture for electrostatically and individually controlling the passage therethrough of each of the micro-ion-beamlets passing through the first electrode;
a third electrode layer in a spaced relation to the second electrode layer and having a third plurality of apertures formed therein and aligned with the first and second plurality of apertures, the third electrode layer having an electrode at each aperture for electrostatically and individually controlling the passage therethrough of each of the micro-ion-beamlets passing through the first electrode and second electrode layer;
wherein the multiplex addressing system is connected to the electrodes in the second and third electrode layers.

8. The pattern generator of claim 7 wherein the first electrode comprises a conductor having the first plurality of apertures formed therein.

9. The pattern generator of claim 8 wherein the second and third electrode layers each comprise an insulator having the second and third plurality of apertures formed therein, and a conductive electrode element at each aperture.

10. The pattern generator of claim 9 wherein the multiplex addressing system further comprises a plurality of row address lines, each row address line connected to each conductive electrode element in a row of apertures of the second electrode layer, and a plurality of column address lines, each column address line connected to each conductive electrode element in a column of apertures of the third electrode layer.

11. A method of producing a focused ion beam comprising a plurality of beamlets in a predetermined pattern, comprising:
generating a plasma;
extracting ions from the plasma through a pattern generator which produces the predetermined pattern of beamlets;
addressing the pattern generator with a multiplex addressing system to produce the predetermined pattern of beamlets;
passing the ions extracted through the pattern generator through aligned apertures in an acceleration and reduction column.

12. The method of claim 1 1 wherein the step of extracting ions through a pattern generator is performed by forming the pattern generator of:
a first electrode positioned adjacent to the ion source and having a first plurality of apertures formed therein for producing an array of micro-ion beamlets by passing ions from the ion source therethrough;
a second electrode layer in a spaced relation to the first electrode and having a second plurality of apertures formed therein and aligned with the first plurality of apertures, the second electrode layer having an electrode at each aperture for electrostatically and individually controlling the passage therethrough of each of the micro-ion-beamlets passing through the first electrode;
a third electrode layer in a spaced relation to the second electrode layer and having a third plurality of apertures formed therein and aligned with the first and second plurality of apertures, the third electrode layer having an electrode at each aperture for electrostatically and individually controlling the passage therethrough of each of the micro-ion-beamlets passing through the first electrode and second electrode layer.

13. The method of claim 12 further comprising connecting the multiplex addressing system to the electrodes in the second and third electrode layers.

14. The method of claim 12 wherein all the electrodes in one row of the second electrode layer are connected to a single row address line of the multiplex addressing system and all the electrodes in one column of the third electrode layer are connected to a single address line of the multiplex addressing system.
